(19) United States
(12) Patent Application Publication
(10) Pub. No.: US 2014/0079746 A1
(43) Pub. Date: Mar. 20, 2014

(54) FULLERENES AND OTHER CAGE MOLECULAR STRUCTURES IN A SPECIAL HYDRATED STATE

(71) Applicant: Grigoriy Vladimirovich Andrievsky, Kharkov (UA)

(72) Inventor: Grigoriy Vladimirovich Andrievsky, Kharkov (UA)

(21) Appl. No.: 13/985,806

(22) PCT Filed: Feb. 9, 2012
(86) PCT No.: PCT/UA2012/000011

§ 371 Date: Nov. 22, 2013

(30) Foreign Application Priority Data
Feb. 16, 2011 (UA) A 2011 01824

(51) Int. Cl. A23L 1/48; A61K 8/02; A61Q 13/00; A61K 9/51

(52) U.S. Cl. CPC A23L 1/48; A61K 9/51; A61K 8/0245; A61Q 13/00
USPC 424/401; 424/490; 512/1; 426/531

(57) ABSTRACT

The invention relates to fullerenes and other fullerene-like caged molecular structures in a special hydrated state. The above-mentioned hydrated compounds can be described by the following general formulae: (xAi@C_{N}B_{jM})@{m(OH)⁻nH₂O}^{m-}mH⁺, (xAi@C_{N}B_{jM})_{y}@{m(OH)⁻nH₂O}^{m-}mH⁺, where x≧0, N>0, M≧0 or x≧0, N≧0, M>0, m >1, n>1, y>1. Furthermore, xAi denotes that x atoms of any chemical elements and/or possible combinations with one another (Ai) are located within a caged structure C_{N}B_{JM}, which may consist of N atoms of carbon and M atoms of any other chemical elements and/or possible combinations Bⱼ thereof. The formula @{m(OH)⁻nH₂O}^{m-}mH+ indicates that the caged structures are enclosed in a shell of m+n molecules of water, and that the caged structure is in a special hydrated state, in which, upon dissociation of the water, the m(OH)⁻ anions thereof are directly bonded to the caged structure and strongly secured on the surface thereof by donor-acceptor bonds.

The invention relates to the special properties of cage molecular structures of different shape, which, while interacting with water (i.e. during their hydration), form robust supramolecular complexes of definite composition with water due to their electron-acceptor properties, geometric form and dimensions. Such complexes, irrespective of the method of their production, can exist only as liquid or solid solutions of cage molecular structures in bulk water.

In particular, such cage molecular structures are fullerenes—hollow and filled sphere-like networks consisting preferably of carbon atoms (see examples infra), which can be used for both research and applied development in different areas of modern nanotechnologies. From the viewpoint of biologically active substances, fullerenes and other cage molecular structures in a special hydrated state can be used in the production of new medicinal, preventive, and hygienic products, perfumes and cosmetics, as well as food industry products, goods for veterinary medicine, agriculture, etc.

The technical solution Aqueous molecular-colloidal solution at least of one hydrated fullerene, RF Patent No. 2213692 dated Oct. 10, 2003 is known, for example, wherein at least one hydrated fullerene is a supramolecular complex with the general formula C_{N}@{m(OH)⁻nH₂O}^{m-} . . . mH⁺, where N≧60, (m+n)≧20, m>1, n>1; and wherein at least part of the H⁺ counter-ions can be substituted with metal ions to the level when the concentration of metal ions in the solution of this complex does not exceed its coagulation threshold.

However, the prior technical solution has several substantial drawbacks due to the fact that it is limited to the class of hydrated hollow cage molecular structures comprising only carbon atoms (C)—fullerene molecules. Thus, it fails to account for the special hydrated state of crystalline and/or amorphous micro and nanosized condensed forms of fullerenes.

The purpose of the invention is both to expand the range of existing and possible cage molecular structures and to account for their condensed forms in hydrated state, wherein, with water molecules, they all form special supramolecular complexes and have supermolecular features, and where, upon being introduced to bulk water, they modify their structural characteristics and properties in a definite manner.

The technical problem proposed is resolved as follows. Fullerenes and other cage molecular structures of the present invention can be described by the general formula xAᵢ@C_{N}B_{jM} (I), wherein xAᵢ means that the x-atoms of any chemical elements and/or their possible combinations with each other (A,) are located inside (denoted by symbol @) of cage structure C_{N}B_{jM}, which can consist of N-atoms of carbon (C) and M-atoms of any other chemical elements and/or their possible mutual combinations (Bⱼ), where the values of x, N and M can correspond to sets of values with x>0, N>0, M=0, or x≧0, N≧0, M>0, as well as y-number of their condensed forms corresponding to the general formula (xAᵢ@C_{N}B_{jM})_{y} (II), upon interaction of their surface with molecules of water, being in aqueous media and depending on the method of preparation of I and II, resulting in the following:

(a) cage molecules I in a hydrated state characterized by the general formula:

(xAᵢ@C_{N}B_{jM})@{m(OH⁻) nH₂O}^{m-}mH⁺ (III), or

(b)condensed forms (crystalline and/or amorphous) of cage molecules II in a hydrated state, having the general formula:

(xAᵢ@C_{N}B_{jM})_{y}@{m(OH⁻)nH₂O}^{m-}mH⁺ (IV), or

(c) combination of III and IV,

wherein m, n, and y can take the values m>1, n>1, and y>1, respectively, and wherein the designation “@{m(OH⁻)nH₂O}^{m-}mH⁺” in formulas III and IV indicates that cage structures I or their condensed forms II are enclosed by the shells comprised of m+n molecules of water, and that I and II are being in a special hydrated state in which, upon dissociation of the water, the mOH⁻ anions thereof are directly bonded and strongly secured on the surface of I and II due to the formation of donor-acceptor bonds, i.e., the bonds that are formed due to the charge transfer.

As seen from the description of the invention's technical substance, it essentially differs from the known prototype, and hence, is a novelty.

The innovative level of the proposed technical solution consists in the following. According to the invention, the proposed technical solution significantly extends the range of objects and embraces both typical fullerenes and other existing or potential cage molecular structures, which can be in a special hydrated state. The invention also accounts for condensed forms:
for which the primary structural elements are single cage molecular structures in a special hydrated state, united with each other (condensed) due to the merging of their hydrated shells,
which are typical crystalline and/or amorphous forms of cage molecular structures and whose physico-chemical nature of surface hydration is similar to that of their primary structural elements.

The invention also accounts for the fact that the uniting property of all proposed molecular structures and their condensed forms in a spatial hydrated state is due to the fact that, when introduced to bulk water, the aforementioned structures and their condensed forms modify the structural characteristics and properties of bulk water in a definite manner.

The invention's practical and commercial applicability has been confirmed by the following data. Having analysed the available scientific and technical information and conducted his own research, the author has obtained the following results:

In 1994, for the first time, the author obtained aqueous solutions of fullerenes C₆₀ and/or C₇₀, with the abbreviated name of FWS. Later, in 1995 to 2005, they were proven to be molecular-colloidal solutions of spherical fractal clusters, the structural unit of which is hydrated fullerene (HyFn) a robust, very hydrophilic supramolecular complex consisting of a fullerene molecule embedded in a spatial multilayer hydrate shell.

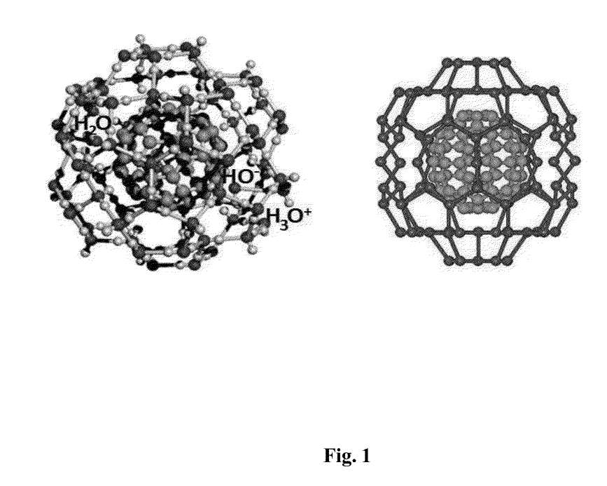

FIG. 1 shows the images of similar supramolecular complexes (III) consisting of a cage molecular structure surrounded by a hydrate shell of closely bound water molecules. In this case, two models are given: III-1 is a hydrated molecule of fullerene C₆₀ [C₆₀@{H₂O}₈₀ ] and III-2 is a hydrated molecule of fullerene C₇₀[C₇₀@{H₂O}₉₀ ] (examples are taken from website: http://www1.1sbu.ac.uk/water/buckmin.html).

A common feature of fullerenes and other cage molecular structures in a spatial hydrated state is that their first closest layer of the closely bound water shell consists of a definite and constant number of water molecules.

In the case of hydrated fullerene C₆₀, designated as C₆₀HyFn with the formula C₆₀@{H₂O}ₙ, its first water layer contains n=22±2 H₂O molecules. With a C₆₀ molecule being 1 nm in size, the diameter of such a fullerene-water complex is 1.6 to 1.8 nm.

In general, the larger the fullerene molecule or other cage molecular structures is in size, a proportionally greater number of water molecules will be accommodated in its first tightly bound hydrate layer of the closely bound water shell.

In addition, water molecules in the first and subsequent layers of closely bound water are rigidly oriented and ordered. The properties of such water layers are similar to those of the liquid crystal state of substances.

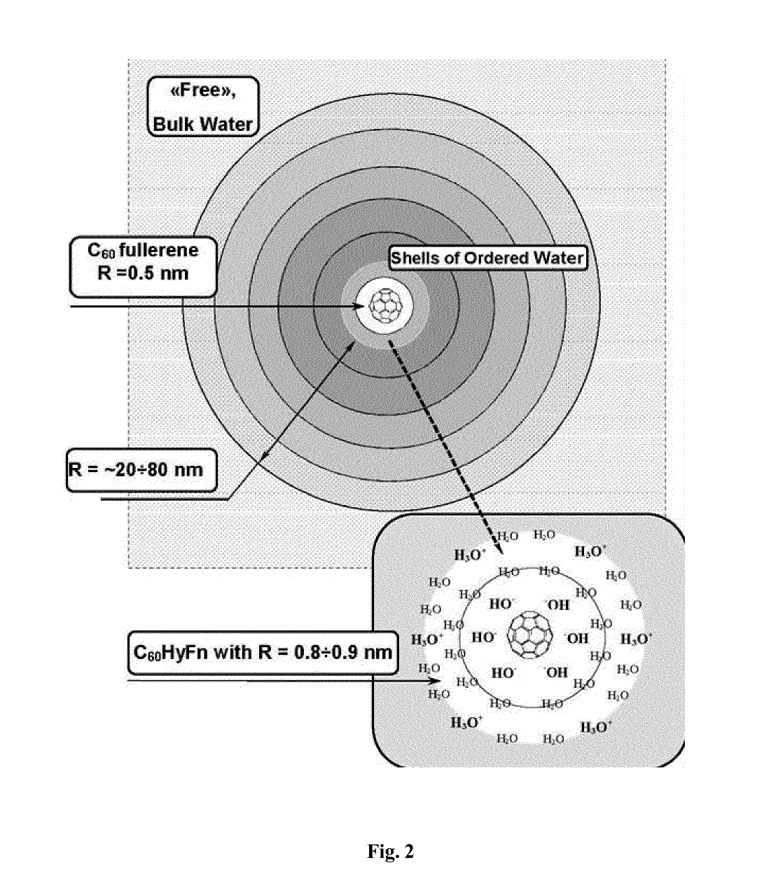

As an example, FIG. 2 shows a state diagram of hydrated fullerene C₆₀ (C₆₀HyFn) in “free” bulk water and surrounding organisation of elongated shells of ordered water molecules, the sizes of which can exceed the diameter of the cage molecule proper, in this case, of C₆₀ (with a radius R˜=0.5 nm), by dozens and hundreds of times. In this regard, the smaller the concentrations of the cage molecular structure are in “free” bulk water in a special hydrated state, the larger the elongated ordered water shells surrounding it will be.

In general, the properties of water in ordered shells fundamentally differ from those of ordinary non-ordered water, ice and water vapour. The state of water in such ordered shells is known as the “fourth state of water” and is called either interfacial water, according to Vladimir Voeikov, or water of exclusion zones, according to Gerald Pollack.

Depending on the conditions in which hydrated structures III and/or hydrated condensed forms IV are found, they can unite mutually into clusters (secondary fractal associates) due to the influence of their hydrate shells. In this regard, the size of the clusters being formed follow a regularly ascending series of values, whereas the sizes of prevailing clusters depend on the concentrations of III and/or IV in the solution. For instance, the less concentrated their solution, the smaller the size of the clusters it contains, and conversely, the more concentrated the solutions of III and/or IV, the bigger their clusters will be.

For instance, in the case of hydrated fullerene C₆₀ (C₆₀HyFn) solutions, the diameters of its spherical clusters will correspond to the series of values (3.4; 7.1; 10.9; 14.5; 18.1; 21.8; 25.4; 28.8; 32.4; 36.0 nm), varying regularly according to the equation: Dᵢ₊₁=Dᵢ+3.4×(10-11%), where i=1, 2, 3, . . . , and D₁=3.4) (G. V. Andrievsky, V. K. Klochkov, E. L. Karyakina, and N. O. Mchedlov-Petrossyan. Studies Of Aqueous Colloidal Solutions Of Fullerene C₆₀ By Electron Microscopy. Chem. Phys. Lett., 300 (1999) 392-396).

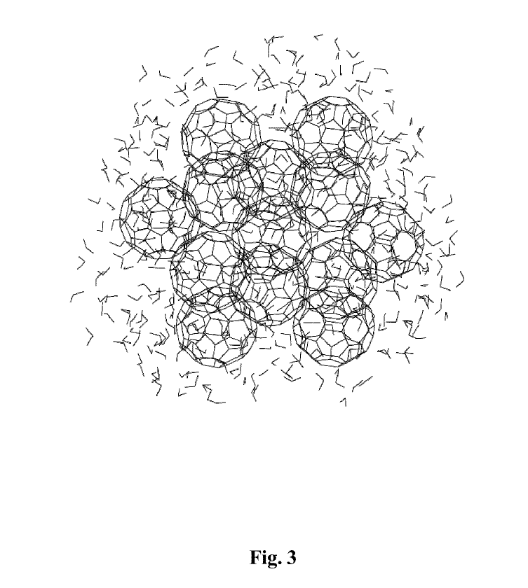

FIG. 3 is an image of the simplest spherical cluster (secondary associate) consisting of several cage molecules (I) in hydrated state (III) or condensed form (II), consisting of y-cage molecules with a spatial hydrate shell on its surface (IV). In this case, this is an example of hydrated icosahedral fullerene C₆₀ nanostructures with the diameter D₁=3.4 nm. They can be presented as two formulas: 13 [C₆₀@{m(OH)⁻nH₂O}^{m-}mH⁺] (for case III) or (C₆₀)₁₃@{m(OH)⁻nH₂O}^{m-}mH⁺ (for case IV).

The common feature of aqueous solutions containing cluster structures (labile associates) III or IV is that, during their dilution or concentration, the clusters are redistributed dynamically by size until establishing an equilibrium state with a definite disperse distribution of clusters typical for given specific conditions (concentration of clusters, ion force magnitude, pH, temperature of their solutions, and other factors).

Also, a common feature of aqueous solutions containing clusters (labile associates) III and/or IV is that the dilution of clusters to definite concentrations results in the full dissociation of clusters so that the diluted solutions obtained contain only non- associated III and/or IV.

In general, the sizes of fullerene or other cage molecular structures in a special hydrated state (III and IV) and/or their clusters in stable aqueous solutions take the values of the above series (3.4; 7.1; 10.9; 14.5; 18.1; 21.8; 25.4; 28.8; 32.4; 36.0 nm). It corresponds to the sizes of supermolecular water structures (its higher-order clusters), into which water molecules themselves are capable of self-organising, this being an inherent property of water.

There are two factors resulting in the special hydrated state of cage molecular structures (I and/or II): (a) the hydrophobic hydration of their surface, and (b) the formation of the first water shell, wherein all water molecules are mutually linked by hydrogen bonds.

This hydrophobic hydration is the result of the electron-acceptor properties of structures I or II, the hydration of which is characterised by these structures, upon interacting with water molecules, forming bonds with them via their oxygen atoms. In this case, water molecules' oxygen atoms act as electron donors, whereas donor-acceptor bonds (bonds with charge transfer), differing in strength, are formed between water molecules and cage structures I or II.

The formation of such donor-acceptor bonds during the hydrophobic hydration of cage structures I or II results in significant modifications of their electron, optical and other physico-chemical characteristics as compared with similar characteristics of structures I or II in the non-hydrated state. (G. V. Andrievsky, V. K. Klochkov, A. Bordyuh, and G. I. Dovbeshko. Comparative Analysis Of Two Aqueous-Colloidal Solutions Of C₆₀ Fullerene With Help Of Ft-Ir Reflectance And Uv-Vis Spectroscopy. Chem. Phys. Letters, 364 (2002) 8-17).

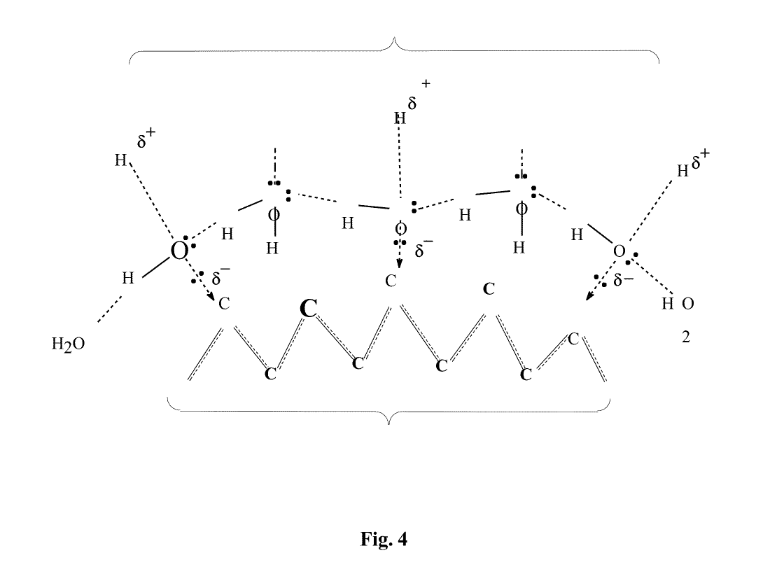

According to the example of hydrated fullerenes C_{N}@{mOH⁻nH₂O}^{m-}mH⁺, FIG. 4 shows a hydration diagram for a section of the cage molecular structure (I or II) surface that is due to: (a) donor-acceptor bonds between water molecules and the cage structure (such bonds are represented as dashed arrows); and (b) hydrogen bonds between water molecules (represented as dashed lines), which unite them all in a unique hydrate shell, embracing the given cage molecular structure.

As compared to ordinary water, for which the acid dissociation index is pκₐ=7, the water molecules involved in the hydrophobic hydration of cage structures I or II, and therewith forming donor-acceptor bonds, very easily dissociate following the reaction: H₂O→H⁺+OH⁻. For example, in the case of hydrated fullerene C₆₀@{mOH⁻nH₂O}^{m-}mH⁺, this reaction occurs over 10⁴ times more effectively than in ordinary water, and where pκₐ=3.5.

Due to this mechanism of the hydrophobic hydration of cage structures I or II, their special hydrated state is manifested by the occurrence therein of acid and cation-exchange properties typical for weak acids (e.g. carbonic acid) and compounds with weakly acid chemical groups, whereas the hydrated surface proper of structures I or II acquires a pronounced negative charge with a ζ (zeta) potential of −10 to −60 mV. (RF Invention Patent No. 2213692 dated Oct. 10, 2003 Aqueous molecular-colloidal solution at least of one hydrated fullerene).

Besides, as a consequence of this mechanism for the hydrophobic hydration of cage structures I or II, their special hydrated state is manifested in that the water melting temperature (kind I phase transition) of the first hydrate shell being lower than 0° C., i.e. lower than the temperature for melting ice. For example, in the case of hydrated fullerene, C₆₀@{mOH⁻nH₂O}^{m-}mH⁺, this temperature is −2.8 ° C.

A common property of cage molecular structures in a special hydrated state, jointly with the elongated ordered water shells organised by them, is that, in their presence, the kinetics and direction of physico-chemical and biochemical processes differ dramatically from the characteristics of similar processes occurring in ordinary, low-ordered bulk water.

The universal antioxidant and radioprotective properties of cage molecular structures in a special hydrated state are a typical example. In general, the antioxidant and radioprotective properties of a chemical antioxidant compound entail its capacity to interact chemically with high-reactivity free radicals (atoms or molecules having one unpaired electron) and to inhibit and suppress chain reactions caused by free radicals.

A distinctive feature of cage molecular structures in a special hydrated state as antioxidants is that they themselves do not participate directly in the specified reactions, whereas their antioxidant, antiradical and radioprotective properties are manifested due to the special properties of the elongated, heterogeneously ordered water shells they organise around themselves (G. V. Andrievsky, V. K. Klochkov, and L. I. Derevyanchenko. Is C₆₀ Fullerene Molecule Toxic?! Fullerenes, Nanotubes and Carbon Nanostructures, 13(4) (2005) 363-376 and G. V. Andrievsky, V. I. Bruskov, A. A. Tykhomyrov, and S. V. Gudkov. Peculiarities Of The Antioxidant And Radioprotective Effects Of Hydrated C₆₀ Fullerene Nanostuctures In Vitro And In Vivo. Free Radical Biology & Medicine, 47 (2009) 786-793).

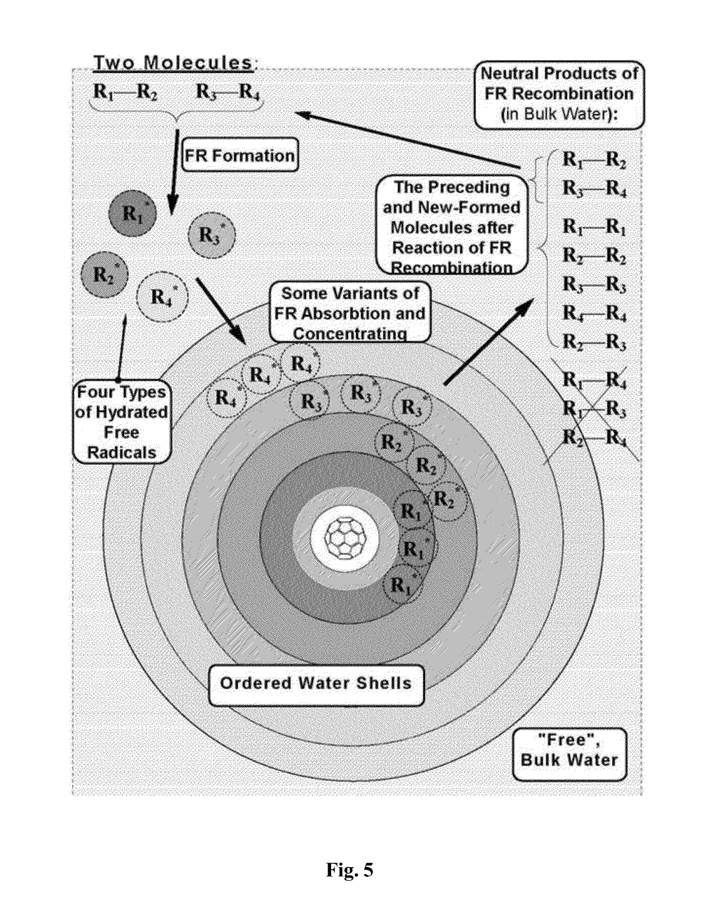

According to the example of hydrated fullerene C₆₀ (C₆₀HyFn), FIG. 5 shows a principal diagram of how, according to recombination reactions (disproportioning), self-destruction occurs and free radicals self-neutralise in an environment of ordered water shells, organised and stabilised by a hydrated cage structure.

The diagram shows that, in the case of non-ordered “free” bulk water, the molecules R₁-R₂ [symbol] R₃-R₄ present therein have, for some reason, formed free radicals FR (R₁*, R₂*, R₃* and R₄*, with the symbol of the unpaired electron “*”). At the same time, it indicates that, being in an aqueous environment and each having its intrinsic chemical and geometric structure, they shall hydrate upon interacting with water, i.e. acquire water shells around themselves with definite properties and spatial structures (in the diagram, different hydrated FR are shown as symbols Rᵢ against circles with different shades of grey).

Further, due to diffusion and according to the principle “similar dissolves in similar”, free radicals FR enter and arrange (are absorbed) in those sites of specially ordered water shells, for which the properties and spatial structures of water are similar to the intrinsic water shells of hydrated FR (in the diagram, similar shells are indicated by the same shades of grey).

Such local accumulation of free radicals from bulk water results in their concentration in specific sites of ordered bulk water. In turn, this increases the probability of their mutual meeting many-fold, as well as the subsequent recombination (disproportioning) reaction. The outcome of such processes is the formation of a multitude of neutral, no-radical molecules. The cage molecular structure proper in a spatial hydrated state does not interact chemically with free radicals. Therefore, chemically it is not modified and expended, and in a unit of time, it is capable of neutralising many hundreds and thousands of free radicals, i.e. it functions as a peculiar catalyst for the recombination of free radicals. Such properties make it akin to those of protein catalysts—enzymes of the intrinsic system for antioxidant protection in live organisms (superoxide dismutase, catalase, peroxydase and others).

The author's research, as well as official preclinical and clinical tests conducted in Ukraine, confirmed the unique antioxidant and radioprotective properties of the cage structure in the form of hydrated fullerene C₆₀. As a result, aqueous solutions of hydrated fullerene C₆₀ were approved for administration by humans as a multifunctional medical and preventive dietary nutrition supplement (Ministry for Healthcare of Ukraine Conclusion No. 05.03.02-04/59179 dated Dec. 2, 2010 and Conclusion No. 05.03.02-04/89993 dated Nov. 19, 2010).

### DESCRIPTION OF EXAMPLES

Typical representatives of the line of fullerenes and other fullerene-identical cage molecular structures (in the formulas, the symbol @ designates that the chemical elements located to its left are encapsulated, i.e. that they are located inside a closed cage structure whose elemental composition is shown to the right of the symbol).

Structures (synthesised or isolated) known to date:
ordinary fullerenes: C₆₀, C₇₀, C₇₄, C₇₆, C₇₈, C₈₀, C₈₂, C₈₄;
endohedral and hetero-fullerenes:

Mₓ@C_{N}, where x=1-3 and N=60-84, and M=H, Li, K, He or atoms of rare earth metals;

C₄₈N₁₂, C₅₇N₃, C₅₉N, (C₅₉N)₂, C₅₉NH;

M₂@C₇₉N , where M=Y; Tb;

C₄₈Si₁₂, C₅₉P—OH, (C₅₉PO)₂;

metal-nitride endofullerenes:

Gd₍ₓ₎Sc₍₃₋ₓ₎N@C₈₀, Gd₃N@C₈₀, Lu₃N@C₈₀;

carbon nano-onions: C_{N}@C_{M};

carbonless: golden “fullerenes” Au₁₆, Au₁₇, Au₁₈.

Structures with a predicted probability of existence:
ordinary fullerenes: C_{N} c 84 <N <540;
endohedral and hetero-fullerenes:

Ng₂@C₆₀, where Ng=He, Ne, Ar, Kr, Xe;

Sc₃N@C₆₇B, Sc₃N@C₆₇N, Sc₃N@C₆₆BN; C₅₀(BN)ₓ, C₂₀B₁₅N₁₅; C₍₆₀₋ₘ₎Si(m), where m≧20;

C₅₆Pt₂, C₅₇Pt₂, C₈₁Pt₂; C₇₀₋ₙPₙ, where n=2 to 10.
carbonless:

“GaP” and “Si”-fullerenes, networks B_{N}, where N=32−56.

As can be seen from the description of the technical essence and examples, the proposed invention intrinsically differs from the prototype, has novel features and an strong invention level, and can be used in the economy, particularly in nanotechnologies for the chemical industry, in the pharmaceutical and medicine sphere, in the food and cosmetics industry, and elsewhere.

1. (canceled)

2. A molecular structure, said molecular structure comprising a caged molecular structure having formula (I):
xAᵢ@C_{N}B_{jM} (I),
wherein xAᵢ denotes x atoms of chemical elements Aᵢ of any kind, or combinations thereof,
@ denotes that said x atoms are located inside of a cage structure C_{N}B_{jM}, said cage structure being comprised of N carbon atoms C and M atoms of other chemical elements Bⱼ, or combinations thereof,
wherein the values of x, N and M belong to a set of values with x>0, N>0, M=0, or to another set of values with x>0, N>0, M>0; or
a condensed form of said caged molecular structure according to formula (II):
(xAᵢ@C_{N}B_{jM})_{y} (II),
wherein y has a value greater than 1; and
wherein said caged molecular structure or said condensed form thereof upon forming surface interactions with water molecules in an aqueous media are capable of forming a caged molecular structure according to formula (I) in a hydrated state, said hydrated state being characterized by formula (III):
(xAᵢ@C_{N}B_{jM})@{m(OH⁻)nH₂O}^{m-}mH⁺ (III),
wherein the values of m and n are each greater than 1.

3. The molecular structure of claim 2, wherein said surface interactions with water molecules in said hydrated state result in formation of a closed hydrate shell, wherein n+m molecules of H₂O are capable of induced dissociation due to formation of donor-acceptor bonds formed by a partial transfer of a negative charge from an oxygen atom of a water molecule to an electron-acceptor surface of said caged molecular structure or said condensed form thereof.

4. The molecular structure of claim 3, wherein m(OH)⁻ anions localise directly on a surface of said hydrated state, causing said caged molecular structure or said condensed form thereof to have a negative surface potential.

5. The molecular structure of claim 4, wherein said m(OH)⁻ anions are united via hydrogen bonding with adjacent water molecules forming an ordered highly polarised water layer.

6. The molecular structure of claim 4, wherein said negative surface potential has a zeta value in a range from about −10 to about −60 mV.

7. The molecular structure of claim 3, wherein a melting temperature of said closed hydrate shell is below 0° C.

8. The molecular structure of claim 3, wherein a diameter of said caged molecular structure in said hydrated state is from about 1.6 to about 1.8 nm.

9. A molecular structure, said molecular structure comprising a caged molecular structure having formula (I):
xAᵢ@C_{N}B_{jM} (I),
wherein xA, denotes x atoms of chemical elements Aᵢ of any kind, or combinations thereof,
@ denotes that said x atoms are located inside of a cage structure C_{N}B_{jM}, said cage structure being comprised of N carbon atoms C and M atoms of other chemical elements Bⱼ, or combinations thereof,
wherein the values of x, N and M belong to a set of values with x>0, N>0, M=0, or to another set of values with x≧0, N≧0, M≧0; or
a condensed form of said caged molecular structure according to formula (II):
(xAᵢ@C_{N}B_{jM})_{y} (II),
wherein y has a value greater than 1; and
wherein said caged molecular structure or said condensed form thereof upon forming surface interactions with water molecules in an aqueous media are capable of forming a condensed crystalline or an amorphous form of a hydrated state of said caged molecular structure, said hydrated state being characterized by formula (IV):
(xAᵢ@C_{N}B_{jM})_{y}@{m(OH⁻)nH₂O}mH⁺ (IV),
wherein the values of m, n, and y are each greater than 1.

10. The molecular structure of claim 9, wherein said surface interactions with water molecules in said hydrated state result in formation of a closed hydrate shell, wherein n+m molecules of H₂O are capable of induced dissociation due to formation of donor-acceptor bonds formed by a partial transfer of a negative charge from an oxygen atom of a water molecule to an electron-acceptor surface of said caged molecular structure or said condensed form thereof.

11. The molecular structure of claim 10, wherein m(OH)⁻ anions localise directly on a surface of said hydrated state, causing said caged molecular structure or said condensed form thereof to have a negative surface potential.

12. The molecular structure of claim 11, wherein said m(OH)⁻ anions are united via hydrogen bonding with adjacent water molecules forming an ordered highly polarised water layer.

13. The molecular structure of claim 11, wherein said negative surface potential has a zeta value in the range from about −10 to about −60 mV.

14. The molecular structure of claim 10, wherein a melting temperature of said closed hydrate shell is below 0° C.

15. The molecular structure of claim 10, wherein a diameter of said caged molecular structure in said hydrated state is from about 1.6 to about 1.8 nm.

16. A molecular structure, said molecular structure comprising a caged molecular structure having formula (I):
xAᵢ@C_{N}B_{jM} (I),
wherein xAᵢ denotes x atoms of chemical elements Aᵢ of any kind, or combinations thereof,
@ denotes that said x atoms are located inside of a cage structure C_{N}B_{jM}, said cage structure being comprised of N carbon atoms C and M atoms of other chemical elements Bⱼ, or combinations thereof,
wherein the values of x, N and M belong to a set of values with x>0, N>0, M=0, or to another set of values with x≧0, N≧0, M>0; or
a condensed form of said caged molecular structure according to formula (II):
(xAᵢ@C_{N}B_{jM})_{y} (II),
wherein y has a value greater than 1; and
wherein said caged molecular structure or said condensed form thereof upon forming surface interactions with water molecules in an aqueous media are capable of forming a combination of a) a caged molecular structure according to formula (I) in a first hydrated state, said first hydrated state being characterized by formula (III):
(xAᵢ@C_{N}B_{jM})@{m(OH⁻)nH₂O}^{m-}mH⁺ (III), and
b) a condensed crystalline or an amorphous forms of said caged molecular structure according to formula (II) in a second hydrated state, said second hydrated state being characterized by formula (IV):
(xAᵢ@C_{N}B_{jM})_{y}@{m(OH⁻)nH₂O}^{m-}mH⁺ (IV),
wherein the values of m, n, and y are each greater than 1.

17. The molecular structure of claim 16, wherein said surface interactions with water molecules in said first hydrated state or said second hydrated state result in formation of a closed hydrate shell, wherein n+m molecules of H₂O are capable of induced dissociation due to formation of donor-acceptor bonds formed by a partial transfer of a negative charge from an oxygen atom of a water molecule to an electron-acceptor surface of said caged molecular structure or said condensed form thereof.

18. The molecular structure of claim 17, wherein m(OH)⁻ anions localise directly on a surface of said first hydrated state or said second hydrated state, causing said caged molecular structure or said condensed form thereof to have a negative surface potential.

19. The molecular structure of claim 18, wherein said m(OH)⁻ anions are united via hydrogen bonding with adjacent water molecules forming an ordered highly polarised water layer.

20. The molecular structure of claim 18, wherein said negative surface potential has a zeta value in the range from about −10 to about −60 mV.

21. The molecular structure of claim 17, wherein a melting temperature of said closed hydrate shell is below 0° C.
